(19) United States
(12) Patent Application Publication
(10) Pub. No.: US 2016/0348047 A1
(43) Pub. Date: Dec. 1, 2016

(54) PRODUCTION OF CIDER WITH PICHIA KLUYVERI YEAST

(71) Applicant: CHR. HANSEN A/S, Hoersholm (DK)

(72) Inventors: Sofie SAERENS, Skovlunde (DK); Jan Hendrik SWIEGERS, Fredensborg (DK)

(73) Assignee: Chr. Hansen A/S, Hoersholm (DK)

(21) Appl. No.: 15/115,162

(22) PCT Filed: Feb. 4, 2015
(86) PCT No.: PCT/EP2015/052237

§ 371 Date: Jul. 28, 2016

(30) Foreign Application Priority Data
Feb. 4, 2014 (EP) 14153806.6

(51) Int. Cl. C12G 3/02; C12P 7/62; A23L 19/00

(52) U.S. Cl. CPC C12G 3/02; A23L 19/00; C12P 7/62; A23V 2002/00

(57) ABSTRACT

The present invention relates to the field of production of a beverage, especially cider, by fermentation of apple juice and/or pear juice. Specifically, the invention relates to a method of preparing a beverage with enhanced levels of desirable fermentation-derived flavor compounds comprising a step of fermentation of apple juice and/or pear juice with at least one Pichia kluyveri yeast strain, a beverage obtainable by the method and a cider comprising a high concentration of the flavor compound, hexyl acetate.

### FIELD OF THE INVENTION

The present invention relates to the field of production of a beverage, especially cider, by fermentation of apple juice and/or pear juice. Specifically, the invention relates to a method of preparing a beverage with enhanced levels of desirable fermentation-derived flavor compounds comprising a step of fermentation of apple juice and/or pear juice with at least one Pichia kluyveri yeast strain, a beverage obtainable by the method and a cider comprising a high concentration of the flavor compound, hexyl acetate.

### BACKGROUND OF THE INVENTION

Aroma volatile compounds are important components of the flavor of fermented beverages, and exert an important influence on final consumer acceptance. In the case of cider, the aroma profile mainly depends on the apple variety used, but also on the choice of yeast used during fermentation. The aroma properties of the fruit used as raw material depend on the combination of volatile compounds present in the fruit, as well as on the concentration and flavor threshold of each volatile. Apple fruit produces many volatile compounds with different chemical structures. For most apple cultivars, esters with fruity notes are quantitatively and qualitatively predominant (Moya-Leon et al. 2007). Apple aroma increases with ripening and the time of harvest is a crucial factor in determining aroma production.

Different apple varieties have been analyzed for fruity ester volatile composition. In Royal Gala apples, butyl and hexyl acetate were the most abundant esters produced, and hexanol, butanol, and 2-methyl butanol were the most abundant higher alcohols found (Moya-Leon et al. 2007). Hexyl acetate, 2-methyl butyl acetate and butyl acetate were found to be major contributors to aroma. The concentrations of these volatiles were higher in late-harvested apples. The same observation was made for Fuji apples, where important contributors for apple aroma were methyl 2-methylbutanoate, isobutyl acetate, ethyl butanoate, butyl acetate and hexyl acetate (Komthong et al. 2007). In another apple variety, called Pink Lady, some of the same volatile ester compounds were measured during maturation: hexyl acetate, hexyl hexanoate, 2-methylbutyl acetate and butyl acetate (Villatoro et al. 2008). These aroma compounds were defined as the prominent aroma compounds that had the highest influence on differentiation of maturity stages. According to the authors, aroma volatile emission is an important factor for definition of fruit ripeness. This is confirmed in several recent articles, which report that ester production is a good indicator for ripeness of apples (Ortiz et al. 2011). The following esters seem the most important volatiles that define a “ripe” apple aroma: hexyl acetate, 2-methylbutyl acetate, hexyl hexanoate and isobutyl acetate.

In a sensory evaluation of character impact components in an apple model mixture, it was very clear that hexyl acetate and trans-2-hexenal are positive contributors that increase “apple” ratings, whereas hexanol is a negative contributor decreasing the “apple” ratings (Bult et al. 2002).

Several Saccharomyces species have been used to ferment apple juice and/or pear juice to cider: Saccharomyces cerevisiae and Saccharomyces uvarum (Leguerinel et al., 1988 & 1989; Scott and O′Reilly, 1996). However, no non-Saccharomyces strains have been used for production of apple cider so far.

Due to the increasing demand for healthier food and beverages, the reduction of ethanol in alcoholic beverages, such as cider, is of considerable commercial interest.

Low-alcohol or alcohol-free ciders are ciders with a low alcohol content or no alcohol which aim to reproduce the full flavor of standard ciders which normally contain at least 4.5% (vol/vol) alcohol.

Low-alcohol cider can be prepared by stopping fermentation by filtration or cooling and then maturation prior to bottling. Low-alcohol or non-alcohol ciders can also be made by producing full-strength cider with an alcohol concentration of above 4.5% (vol/vol) and then removing the alcohol by processes such as boiling off the alcohol or reverse osmosis. In some cases ciders with low alcohol contents are made simply by diluting full-strength ciders with water and/or apple/pear juice.

As these processes result in evaporation, loss or dilution of fermentation-derived flavor compounds, these types of ciders are often characterized as being less flavorful than full-strength cider or may contain high concentrations of undesirable flavors, such as hexanol which is present in different concentrations in apples of different varieties and maturation stage.

European patent application EP2641962 relates to the use of a Pichia kluyveri strain for fermentation of orange juice to produce a drink with a low alcohol content.

There is no mention of the use of Pichia kluyveri for fermentation of apple juice and/or pear juice.

There exists a need for alternative methods of producing beverages based on the fermentation of apple juice and/or pear juice wherein the presence of desirable flavor compounds, such as hexyl acetate and isobutyl acetate, is enhanced.

Furthermore, there exists a need for methods for preparing a low-alcohol or alcohol-free fermented beverage based on apple juice and/or pear juice with better organoleptic properties.

### SUMMARY OF THE INVENTION

It has unexpectedly been found that the fermentation of apple juice with Pichia kluyveri results in a very fresh cider product. Especially the main compounds regarded as giving “fresh apple flavor” were enhanced.

Since Pichia kluyveri consumes the sugar to produce flavor compounds with little ethanol produced, this method can be used to prepare non-alcohol or low-alcohol cider with increased amounts of hexyl acetate and other desirable flavor compounds.

When a higher percentage of alcohol is wanted, a longer fermentation with Pichia kluyveri and/or sequential fermentation with a Saccharomyces yeast can be performed.

Accordingly, an aspect of the present invention relates to a method of preparing a beverage comprising the steps of
a) providing an apple juice and/or pear juice; and
b) fermenting the apple juice and/or pear juice with at least one Pichia kluyveri yeast strain to obtain the beverage.

Another aspect of the present invention is directed to a beverage obtainable by the method according the previous aspect of the invention.

A further aspect relates to a cider having a hexyl acetate concentration of at least 0.7 ppm.

### BRIEF DESCRIPTION OF THE DRAWINGS

FIG. 1 illustrates isobutyl acetate concentrations in 3 apple juices fermented with Pichia kluyveri strain PK-KR1 (juice with Pichia) or not fermented (con juice).

FIG. 2 depicts isoamyl acetate concentrations in 3 apple juices fermented with Pichia kluyveri strain PK-KR1 (juice with Pichia) or not fermented (con juice).

FIG. 3 shows ethyl propionate concentrations in 3 apple juices fermented with Pichia kluyveri strain PK-KR1 (juice with Pichia) or not fermented (con juice).

FIG. 4 illustrates ethyl valerate concentrations in 3 apple juices fermented with Pichia kluyveri strain PK-KR1 (juice with Pichia) or not fermented (con juice).

FIG. 5 depicts hexanol concentrations in 3 apple juices fermented with Pichia kluyveri strain PK-KR1 (juice with Pichia) or not fermented (con juice).

FIG. 6 shows hexyl acetate concentrations in 3 apple juices fermented with Pichia kluyveri strain PK-KR1 (juice with Pichia) or not fermented (con juice).

### DETAILED DESCRIPTION OF THE INVENTION

The inventors of the present invention have unexpectedly found that Pichia kluyveri yeast strains, when used to ferment apple juice, are able to increase the presence of desirable flavor compounds in the fermented beverages. In particular, the flavor compound most associated with fresh apple flavor and aroma were found to be increased.

The process of preparing cider is well known to the skilled person and can be outlined in the following way; apple juice and/or pear juice is prepared by washing the fruits, optionally milling the fruits into pulp and extracting (preferably by pressing) the juice of the fruits without the pulp. Fermentation of the fruit juice is normally carried out either by spontaneous fermentation by indigenous yeast or by controlled fermentation in sterile settings by addition of cultured yeast but a combination of spontaneous fermentation and the addition of cultured yeast can also occur. After fermentation the cider is racked and the cider is optionally filled on bottles and optionally pasteurized.

During this process the individual cider maker may choose to exercise several other options, such as but not limited to the addition of pectinase during milling/pressing, adjustment of pH, addition of sulphur dioxide as an antimicrobial agent, malolactic fermentation, maturation in tanks or oak vats, fining and filtration, in order to achieve the desired end-prod uct.

The term “cider” as used herein refers to the alcohol-free, low-alcohol or alcoholic beverage derived from apple juice (apple cider) or pear juice (perry cider) or blends of apple juice and pear juice by adding a yeast strain to ferment the apple juice and/or pear juice.

The term “alcohol-free beverage” herein is defined as a liquid for drinking with an alcohol content of no more than 0.5% (vol/vol) of alcohol.

The term “low-alcohol beverage” herein is defined as a liquid for drinking with an alcohol content of more than 0.5% (vol/vol) of alcohol and no more than 1.2% (vol/vol) of alcohol.

The term “alcoholic beverage” herein is defined as a liquid for drinking with an alcohol 5 content of more than 1.2% (vol/vol) of alcohol.

The method of preparing a beverage of the present invention comprises the steps of
a) providing an apple juice and/or pear juice; and
b) fermenting the apple juice and/or pear juice with at least one Pichia kluyveri yeast strain to obtain the beverage.

In a preferred embodiment step b) comprises fermenting the apple juice and/or pear juice with at least one Pichia kluyveri yeast strain followed by fermenting the apple juice and/or pear juice with at least one Saccharomyces yeast strain.

Saccharomyces yeast strains include but is not limited to yeast of the species Saccharomyces cerevisiae, Saccharomyces bayanus, Saccharomyces paradoxus, Saccharomyces pastorianus, Saccharomyces cariocanus, Saccharomyces kudriavzevii, Saccharomyces mikitae, Saccharomyces arboricolus and Saccharomyces uvarum.

In a preferred embodiment the Saccharomyces yeast strain is selected from the group consisting of a Saccharomyces cerevisiae yeast strain, a Saccharomyces bayanus yeast strain and a Saccharomyces uvarum yeast strain.

In another preferred embodiment step b) comprises fermenting the apple juice and/or pear juice with at least one Pichia kluyveri yeast strain and allowing spontaneous fermentation of the apple juice and/or pear juice to take place.

The onset of spontaneous fermentation can readily be determined by the skilled person as CO₂ will start to form quickly and the concentration of ethanol will increase at a rate of at least 1% (vol/vol) per day.

Preferably, the fermentation of the apple juice and/or pear juice with the at least one Pichia kluyveri yeast strain is carried out for at least 12 hours, such as for at least 18 hours, such as for at least 24 hours, such as for at least 2 days, such as for at least 4 days, such as for at least 7 days.

In a preferred embodiment the fermentation of the apple juice and/or pear juice with the at least one Pichia kluyveri yeast strain is carried out at a temperature of between 4-35° C. In a more preferred embodiment the fermentation of the apple juice and/or pear juice with the at least one Pichia kluyveri yeast strain is carried out at a temperature of between 18-22⁰C.

In another preferred embodiment the Pichia kluyveri yeast strain is inoculated in a 5 concentration of at least 1×10⁴ CFU/ml, such as at least 5×10⁴ CFU/ml, such as at least 1×10⁵ CFU/ml, such as at least 5×10⁵ CFU/ml, such as at least 1×10⁶ CFU/ml, such as at least 5×10⁶ CFU/ml.

In yet another preferred embodiment the method further comprises the step c) bottling and bottle pasteurizing the beverage.

In an even further preferred embodiment the at least one Pichia kluyveri strain is selected from the group consisting of the Pichia kluyveri PK-KR1 (JT1.28 or strain A) strain that was deposited on 24 August 2006 at the National Measurement Institute, 541-65 Clarke Street, South Melbourne, Victoria 3205, Australia, by University of Auckland, School of Biological Sciences, Auckland 1142, New Zealand, under accession numbers V06/022711, and the Pichia kluyveri PK-KR2 (JT3.71) that was deposited on 24 August 2006 at the National Measurement Institute, 541-65 Clarke Street, South Melbourne, Victoria 3205, Australia, by University of Auckland, School of Biological Sciences, Auckland 1142, New Zealand, under accession numbers V06/022712.

As discussed herein and as exemplified in Example 1, the method according to the present invention is very useful for preparing alcohol-free or low-alcohol beverages with increased levels of desirable fermentation-derived flavor compounds.

Thus, in a preferred embodiment, the beverage has an alcohol content of at the most 4.5% (vol/vol), such as at the most 1.2% (vol/vol).

Preferably, the fermentation is carried out in the absence of Saccharomyces yeast, such as with Pichia kluyveri yeast as the only yeast present.

Furthermore it is preferred that the fermentation of the apple juice and/or pear juice with the at least one Pichia kluyveri yeast strain is carried out for a maximum of 5 days.

The fermentation may be stopped by any suitable method known to the skilled person, including but not limited to chilling the beverage to a temperature below 4° C., filtration, pasteurization and centrifugation.

Alternatively, for producing an alcoholic beverage it is preferred to subsequently either perform controlled fermentation of the beverage with a Saccharomyces yeast strain or allow for spontaneous fermentation of the beverage (see Example 2).

Thus, in a further embodiment the method comprises a further step c) fermenting the apple juice and/or pear juice with a Saccharomyces yeast strain to obtain the beverage.

In a further aspect the present invention is directed to a beverage obtainable by the method according to any of the preceding claims.

As can be seen from Examples 1 and 2 herein, the fermentation of apple juice with Pichia kluyveri increases significantly the amount of hexyl acetate in the fermented apple juice.

Thus, in a preferred embodiment the beverage has a hexyl acetate concentration of at least 0.7 ppm, such as at least 0.8 ppm, such as at least 0.9 ppm, such as at least 1.0 ppm, such as at least 1.1 ppm, such as at least 1.2 ppm, such as at least 1.3 ppm, such as at least 1.4 ppm, such as at least 1.5 ppm, such as at least 1.6 ppm, such as at least 1.7 ppm, such as at least 1.8 ppm, such as at least 1.9 ppm, such as at least 2.0 ppm.

Example 1 shows that fermentation of apple juice with Pichia kluyveri also increases significantly the amount of isobutyl acetate in the fermented apple juice.

Thus, in another preferred embodiment the beverage has an isobutyl acetate concentration of at least 0.1 ppm, such as at least 0.15 ppm, such as at least 0.2 ppm, such as at least 0.25 ppm.

In yet a further aspect, the present invention is directed to a cider having a hexyl acetate concentration of at least 0.7 ppm, such as at least 0.8 ppm, such as at least 0.9 ppm, such as at least 1.0 ppm, such as at least 1.1 ppm, such as at least 1.2 ppm, such as at least 1.3 ppm, such as at least 1.4 ppm, such as at least 1.5 ppm, such as at least 1.6 ppm, such as at least 1.7 ppm, such as at least 1.8 ppm, such as at least 1.9 ppm, such as at least 2.0 ppm.

In a preferred embodiment the cider has an isobutyl acetate concentration of at least 0.1 ppm, such as at least 0.15 ppm, such as at least 0.2 ppm, such as at least 0.25 ppm.

In a further preferred embodiment the cider has an alcohol concentration of at the most 4.5% (vol/vol), such as at the most 1.2% (vol/vol).

In another preferred embodiment the beverage is an apple cider.

### EXAMPLES

Materials and Methods

Fermentation Set-Up

Lab-scale fermentation trials were carried out in 500 ml or 2 L of apple juice. Fermentation was performed for 24 hours or 7 days, as indicated, at 20° C. All yeast strains were inoculated at 5 million cells per ml.

Headspace GC-FID Analysis

Headspace gas chromatography coupled with flame ionization detection (GC-FID) was used for the measurement of esters and higher alcohols in the fermentation products. Fermentation samples were centrifuged, after which 2 ml was collected in vials. Samples were then analyzed with a calibrated Perkin Elmer GC System with a headspace sampler. The GC was equipped with a DB-WAXETR column (length, 30 m; internal diameter, 0.25 mm; layer thickness, 0.5 μm; Agilent Technologies, Germany). The split-splitless injector was used and held at 180° C. Samples were heated for 30 min at 70° C. in the headspace autosampler before injection (needle temperature: 110° C.). Helium was used as the carrier gas. After starting at 60° C., the oven temperature was raised after 2 min from 60° C. to 230° C. at 45° C/min and was finally held at 230° C. for 5 min. During the GC-program a constant flow rate (10 mL/min) of the carrier gas (He) was maintained. The FID temperature was kept constant at 220° C. respectively. The results were analyzed with Turbochrom software.

Ethanol and Sugar Analysis

Ethanol and sugar analysis was carried out with OenofossTM equipment according to the manufacturer's protocol.

Results

### Example 1

### Production of Alcohol-Free Cider with Pichia kluyveri

Three different apple juices were fermented with a Pichia kluyveri yeast strain for the production of an alcohol-free cider:
apple juice made from concentrate A
apple juice made from concentrate B
fresh apple juice

2 liter of apple juice was fermented with Pichia kluyveri strain PK-KR1 for 24 hours at 20° C. After 24 hours, samples were taken for flavor analysis to investigate the flavor profile of the fermented juices, compared with the control, where no yeast was added. To measure how much sugar was degraded into ethanol, sugar and ethanol concentration was measured before and after fermentation (Table 1).

TABLE 1
Sugar and ethanol levels of the different apple juices.

| | Sugar level | Sugar (°Brix) | Ethanol (% v/v) |
|---|---|---|---|
| | (°Brix) before | level after | after |
| Juice | fermentation | fermentation | fermentation |
| Apple juice (concA) | 11.1 | 10.8 | 0.2 |
| Apple juice (concB) | 12.1 | 11.4 | 0.3 |
| Fresh apple juice | 12.2 | 11.8 | 0.2 |

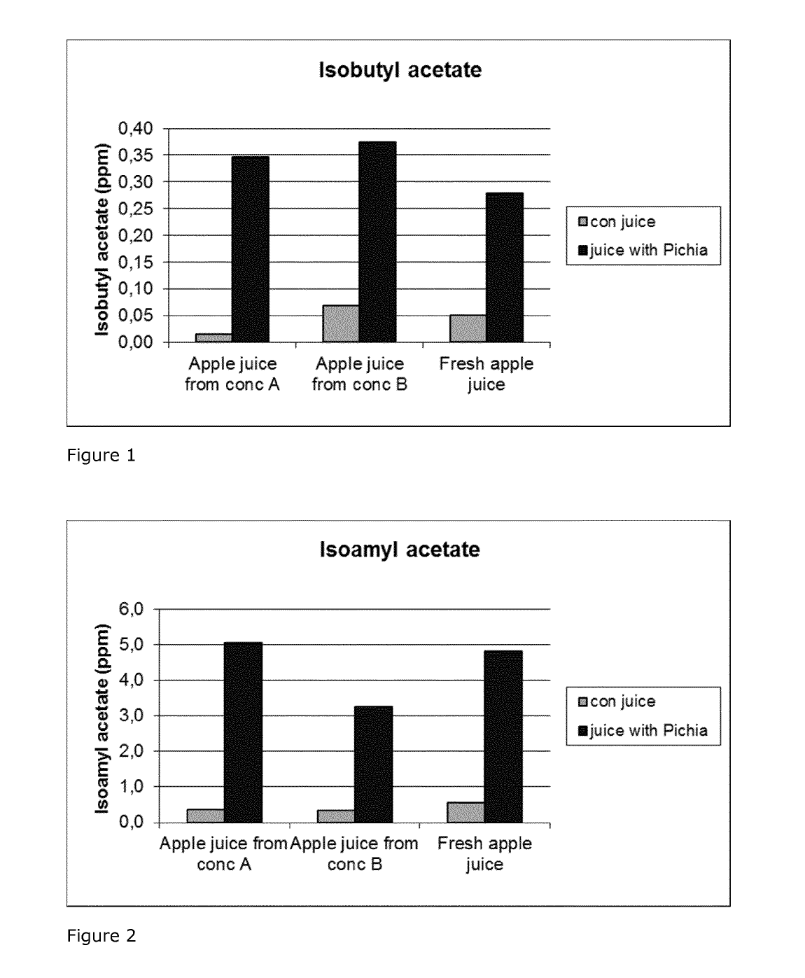

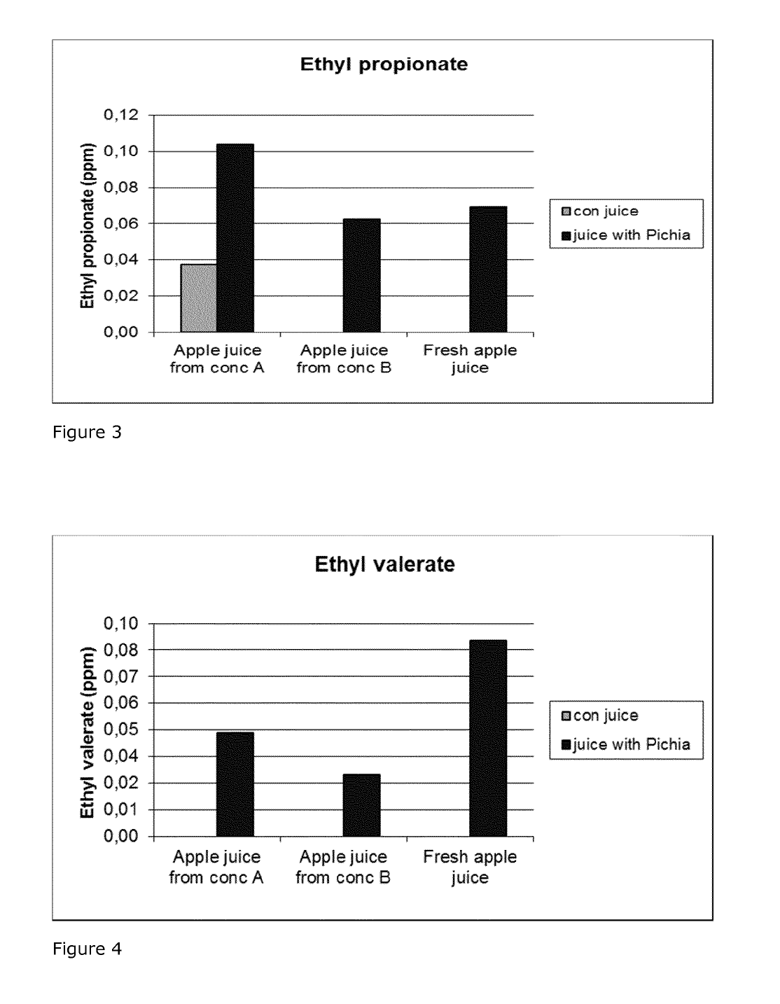

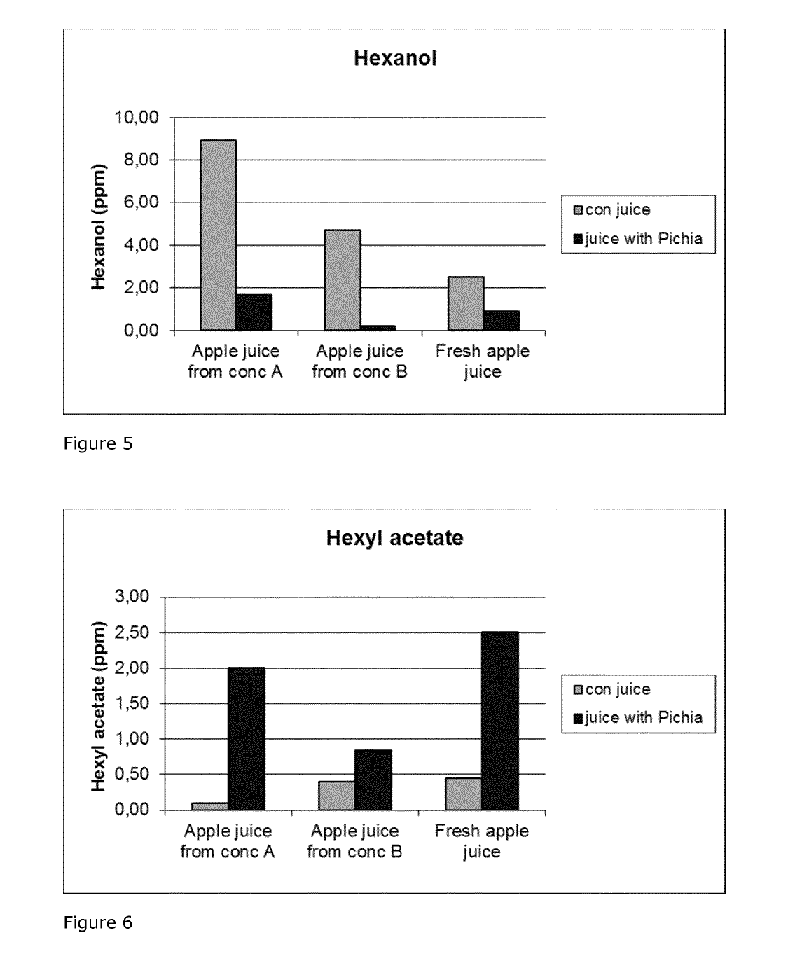

Flavor compounds were measured with headspace-GC-FID and the amounts of 6 compounds were found to be different (see FIGS. 1 to 6) in the fermented test juices as compared to the controls. These compounds were isobutyl acetate, isoamyl acetate, ethyl propionate, valerate, hexanol and hexyl acetate. The amounts of ethyl butyrate and acetaldehyde did not significantly differ.

As can be seen from the results, adding Pichia kluyveri to the apple juice increased the production of isobutyl acetate, isoamyl acetate, ethyl propionate, ethyl valerate, and hexyl acetate. Hexanol is a precursor for hexyl acetate and this compound was very efficiently converted to hexyl acetate by the Pichia kluyveri strain, so we can see a clear decrease in hexanol and increase in hexyl acetate concentration.

Furthermore, Pichia kluyveri did not ferment a lot of sugar during the 24 hours, as only 0.2 to 0.3% of ethanol was formed. In this way, Pichia kluyveri seems to be an excellent yeast strain for fermenting juice without production of ethanol. Sensory analysis of the fermented apple juices revealed that the addition of Pichia kluyveri increased the fresh apple flavor of the apple juice and made the apple juice from concentrate smell like fresh apple juice. This means fresh apple flavor was enhanced in the apple juice from concentrate.

### Example 2

### Production of Alcoholic Cider with Pichia kluvyeri, Torulaspora delbrueckii and Kluyveromyces thermotolerans

To compare Pichia kluyveri with two other non-Saccharomyces yeast strains, Torulaspora delbrueckii and Kluyveromyces thermotolerans, cider from fresh apple juice was produced. In this experiment, the goal was to produce an alcoholic cider, as spontaneous fermentation was allowed to finish the fermentation (the apple juice was not pasteurized). The fresh apple juice was fermented at 20° C. for 7 days and consisted of a mix of Belle de Boskoop, James Greaves, Cox Orange, Filipa and Cox Pomona apples. In all cases, a spontaneous fermentation started 2 days after addition of the non-Saccharomyces yeast strains (the rate of increase of the ethanol concentration reached at least 1% (vol/vol) per day) and finished the fermentation (Table 2).

TABLE 2
Final cider parameters.

| | | Ethanol % | |
|---|---|---|---|
| | TA (g/L) | (v/v) | VA (g/L) |
| Pichia kluyveri | 5.6 | 7.6 | 0.3 |
| Torulaspora delbrueckii | 5.3 | 7.6 | 0.37 |
| Kluyveromyces thermotolerans | 5.7 | 7.8 | 0.48 |
| Spontaneous fermentation | 5.8 | 7.6 | 0.28 |
| Apple juice | 4.4 | 0.6 | 0.55 |

TA = Total acidity
VA = Volatile acidity

Final hexyl acetate and hexanol concentrations are given in Table 3.

TABLE 3
Hexyl acetate and hexanol concentrations in final cider
produced with different yeast strains.

| | Hexyl acetate (ppm) | Hexanol (ppm) |
|---|---|---|
| Pichia kluyveri | 1.07 | 3.46 |
| Torulaspora delbrueckii | 0.63 | 3.24 |
| Kluyveromyces thermotolerans | 0.40 | 3.63 |
| Spontaneous fermentation | 0.54 | 3.80 |
| Apple juice | 0.26 | 6.29 |

From Table 3, it is clear that the cider produced with Pichia kluyveri produces the highest amount of hexyl acetate, compared to the two other non-Saccharomyces strains. This means that using Pichia kluyveri for cider production is the best way to increase hexyl acetate in the final product. All yeast strains enhance the hexyl acetate concentration, compared to the basic apple juice, but Pichia kluyveri produces the highest amount in the final product. The hexanol concentration is more or less similar in all cider products, but the hexyl acetate/hexanol ratio is higher with Pichia kluyveri, resulting in an increased fresh apple flavor in the final product. Table 2 shows that all ciders have similar final ethanol concentrations, meaning that this technique can be used to produce extra flavor in an alcoholic cider as well. In this case, a sequential inoculation is needed with first a Pichia kluyveri yeast strain, followed by inoculation of a Saccharomyces sp. yeast strain or a spontaneous fermentation.

### CONCLUSION

It is very clear from the results that the use of Pichia kluyveri can enhance the fresh apple flavor in apple juice, making it suitable to use for both alcohol-free and alcoholic cider production. Especially the high production of hexyl acetate with Pichia kluyveri is very surprising. This compound is the most important flavor compound in cider determining fresh apple flavor. To produce this flavor compound, Pichia kluyveri uses hexanol as substrate, which is another very positive factor, as hexanol is regarded as a bad flavor compound in apple juice, resulting in a green astringent apple flavor and bad apple ratings. Pichia kluyveri does not produce ethanol when it is fermented completely anaerobic, as the yeast is using all the sugar to produce flavor compounds. In this way, Pichia kluyveri can be used to produce alcohol-free cider, or can be combined with a Saccharomyces yeast to produce cider with fresh apple flavor.

Deposits

The Pichia kluyveri PK-KR1 (JT1.28 or strain A) and PK-KR2 (JT3.71) strains were deposited on 24 August 2006 at the National Measurement Institute, 51-65 Clarke Street, South Melbourne, Victoria 3205, Australia, by University of Auckland, School of Biological Sciences, Auckland 1142, New Zealand, and given the accession numbers V06/022711 and V06/022712 as described in WO 2009/110807.

### REFERENCES

Bult, J. H., Schifferstein, H. N., Roozen, J. P., Boronat, E. D., Voragen, A. D., and Kroeze, J. H. (2002) Sensory evaluation of character impact components in an apple model mixture. Chemical Senses, 27:485-94.

Komthong, P., Hayakawa, S., Katoh, T., Igura, N., and Shimoda, M. (2006) Determination of potent odorants in apple by headspace gas dilution analysis. In: LWT—Food Science and Technology, 39:472-478.

Leguerinel, I., Mafart, P., Cleret, J. J., and Bourgeois, C. (1988) Yeast strain and the formation of flavor components in cider. Journal of the Institute of Brewing, 96:391-395.

Leguerinel, I., Mafart, P., Bourgeois, C. and Cleret, J. J. (1989) Yeast strain and kinetic aspects of the formation of flavour components in cider. Journal of the Institute of Brewing, 95:405-409.

Moya-Leon, M. A., Vergara, M., Bravo, C., Pereira, M. and Moggia C. (2007) Development of aroma compounds and sensory quality of ‘Royal Gala’ apples during storage. Journal of Horticultural Science and Biotechnology 82:403-413.

Ortiz, A., Graell, J., Lara, I. (2011) Volatile ester-synthesizing capacity throughout on-tree maturation of ‘Golden Reinders’ apples. Scientia Horticulturae, 131:6-14.

Villatoro, C., Altisent, R., Echeverria, G., Graell, J., Lopez, M .L., and Lara, I. (2008) Changes in biosynthesis of aroma volatile compounds during on-tree maturation of ‘Pink Lady®’ apples.

1. A method of preparing a beverage comprising the steps of
a) providing an apple juice and/or pear juice; and
b) fermenting the apple juice and/or pear juice with at least one Pichia kluyveri yeast strain to obtain the beverage.

2. The method according to claim 1, wherein the fermentation of the apple juice and/or pear juice with the at least one Pichia kluyveri yeast strain is carried out for at least 12 hours.

3. The method according to claim 1, wherein the fermentation of the apple juice and/or pear juice with the at least one Pichia kluyveri yeast strain is carried out at a temperature of between 4-35° C.

4. The method according to claim 3, wherein the fermentation of the apple juice and/or pear juice with the at least one Pichia kluyveri yeast strain is carried out at a temperature of between 18-22° C.

5. The method according to claim 1, further comprising the step c) bottling and bottle pasteurizing the beverage.

6. The method according to claim 1, wherein the at least one Pichia kluyveri strain is selected from the group consisting of the Pichia kluyveri PK-KR1 (JT 1.28 or strain A) strain deposited at the National Measurement Institute, 541-65 Clarke Street, South Melbourne, Victoria 3205, Australia, under accession number V06/022711, and the Pichia kluyveri PK-KR2 (JT3.71) strain that was deposited at the National Measurement Institute, 541-65 Clarke Street, South Melbourne, Victoria 3205, Australia, under accession numbers number V06/022712.

7. The method according to claim 1, wherein the beverage has an alcohol content of at the most 4.5% (vol/vol).

8. The method according to claim 7, wherein the beverage has an alcohol content of at the most 1.2% (vol/vol).

9. The method according to claim 7, wherein the fermentation of the apple juice and/or pear juice with the at least one Pichia kluyveri yeast strain is carried out for a maximum of 5 days.

10. The method according to claim 1 further comprising fermenting the apple juice and/or pear juice with at least one Saccharomyces yeast strain to obtain the beverage.

11. A cider which has a hexyl acetate concentration of at least 0.7 ppm.

12. The cider according to claim 11 which has an isobutyl acetate concentration of at least 0.1 ppm.

13. The cider according to claim 11 which has an alcohol concentration of at the most 4.5% (vol/vol) alcohol.

14. The cider according to claim 13 which has an alcohol concentration of at the most 1.2% (vol/vol).
